(19) United States
(12) Patent Application Publication
(10) Pub. No.: US 2015/0157882 A1
(43) Pub. Date: Jun. 11, 2015

(54) METHOD OF CONTROLLING TEMPERATURE OF TISSUE AND TEMPERATURE CONTROLLING APPARATUS USING THE METHOD

(71) Applicant: SAMSUNG ELECTRONICS CO., LTD., Suwon-si (KR)

(72) Inventors: Sanghyun KIM, Hwaseong-si (KR); Hotaik LEE, Yongin-si (KR); Wonchul BANG, Seongnam-si (KR)

(73) Assignee: SAMSUNG ELECTRONICS CO., LTD., Suwon-si (KR)

(21) Appl. No.: 14/564,608

(22) Filed: Dec. 9, 2014

(30) Foreign Application Priority Data
Dec. 10, 2013 (KR) 10-2013-0153208

(51) Int. Cl. A61N 7/02

(52) U.S. Cl. CPC A61N 7/02

(57) ABSTRACT

Provided are a method of controlling a temperature of tissue and a temperature controlling apparatus using the method. The method includes measuring a temperature of a target tissue, determining an intensity of ultrasound irradiation with respect to the target tissue based on a target temperature of the target tissue and the measured temperature of the target tissue, and irradiating the ultrasound irradiation having the determined intensity to the target tissue.

### CROSS-REFERENCE TO RELATED APPLICATIONS

This application claims the benefit of Korean Patent Application No. 10-2013-0153208, filed on Dec. 10, 2013, in the Korean Intellectual Property Office, the disclosure of which is incorporated herein in its entirety by reference.

### BACKGROUND

1. Field

The exemplary embodiments relate to methods of controlling a temperature of tissue and temperature controlling apparatuses by using the method.

2. Description of the Related Art

With advances in medicine, minimally invasive surgery has recently been replaced with noninvasive surgery for the local treatment of a tumor. An example of a non-invasive surgery method for local treatment of a tumor is a high intensity focused ultrasound (HIFU) method.

When HIFU is applied to tissue, a temperature of the tissue is increased by thermal energy due to the HIFU.

### SUMMARY

Exemplary embodiments provide methods of controlling a temperature of tissue, in which optimum ultrasound intensity for maintaining a target tissue at a target temperature is determined and an ultrasound of the optimum ultrasound intensity is irradiated to the target tissue, and temperature controlling apparatuses which use the methods.

Additional aspects will be set forth in part in the description which follows and, in part, will be apparent from the description, or may be learned by practice of the presented exemplary embodiments.

According to an aspect of an exemplary embodiment, there is provided a method of controlling a temperature of tissue, including: measuring a temperature of a target tissue; determining an intensity of ultrasound irradiation with respect to the target tissue based on a target temperature of the target tissue and the measured temperature of the target tissue; and irradiating the ultrasound irradiation having the determined intensity to the target tissue.

According to another aspect of an exemplary embodiment, there is provided a temperature controlling apparatus including: a temperature measurer configured to measure a temperature of the target tissue; a temperature controller configured to determine an intensity of ultrasound irradiation with respect to the target tissue based on a target temperature of the target tissue and the measured temperature of the target tissue; and an ultrasound irradiator configure to irradiate the ultrasound irradiation having the determined intensity to the target tissue.

### BRIEF DESCRIPTION OF THE DRAWINGS

These and/or other aspects will become apparent and more readily appreciated from the following description of the exemplary embodiments, taken in conjunction with the accompanying drawings in which:

FIG. 1 is a block diagram illustrating a structure of a temperature controlling apparatus according to an exemplary embodiment;

FIG. 2 is a diagram to describe a temperature control unit of a temperature controlling apparatus according to an exemplary embodiment;

FIG. 3 illustrates a method of determining a position of a target tissue, to which ultrasound is to be irradiated, the method being performed by using an irradiation position determination module of the temperature control unit, according to an exemplary embodiment;

FIG. 4 shows graphs showing a change in temperature of a plurality of foci on which ultrasound is focused with respect to a target tissue, according to time, when a temperature control with respect to the target tissue is performed by using a temperature controlling apparatus according to an exemplary embodiment;

FIG. 5 illustrates a temperature distribution with respect to a target tissue when a temperature control with respect to the target tissue is performed by using a temperature controlling apparatus according to an exemplary embodiment;

FIG. 6 is a flowchart illustrating a method of controlling a temperature of tissue, according to an exemplary embodiment; and

FIG. 7 is a detailed flowchart illustrating an operation of determining an intensity of ultrasound irradiation in a method of controlling a temperature of tissue, according to an exemplary embodiment.

### DETAILED DESCRIPTION

Reference will now be made in detail to exemplary embodiments, examples of which are illustrated in the accompanying drawings, wherein like reference numerals refer to the like elements throughout. In this regard, the exemplary embodiments may have different forms and should not be construed as being limited to the descriptions set forth herein. Accordingly, the exemplary embodiments are merely described below, by referring to the figures, to explain aspects of the exemplary embodiments. Expressions such as “at least one of,” when preceding a list of elements, modify the entire list of elements and do not modify the individual elements of the list.

Hereinafter, the exemplary embodiments will be described in detail with reference to the attached drawings based on examples that are just for illustration, without limiting the exemplary embodiments. The following descriptions of the exemplary embodiments do not limit or define the scope of the exemplary embodiments. Details that are easily derivable by one of ordinary skill in the art to which the exemplary embodiments pertain based on the detailed description are construed as being in the scope of the exemplary embodiments.

In the present specification, the terms such as “comprise” or “include” should not be construed as necessarily including various elements or processes described in the specification, and it should be construed that some of the elements or the processes may not be included, or additional elements or processes may be further included.

In the present description, terms including ordinal numbers such as ‘first’, ‘second’, etc. are used to describe various elements but the elements should not be defined by these terms. The terms are used only for distinguishing one element from another element.

The exemplary embodiments relate to a method of controlling a temperature of tissue and a temperature controlling apparatus using the method. Descriptions that are well known to one of ordinary skill in the art will be omitted.

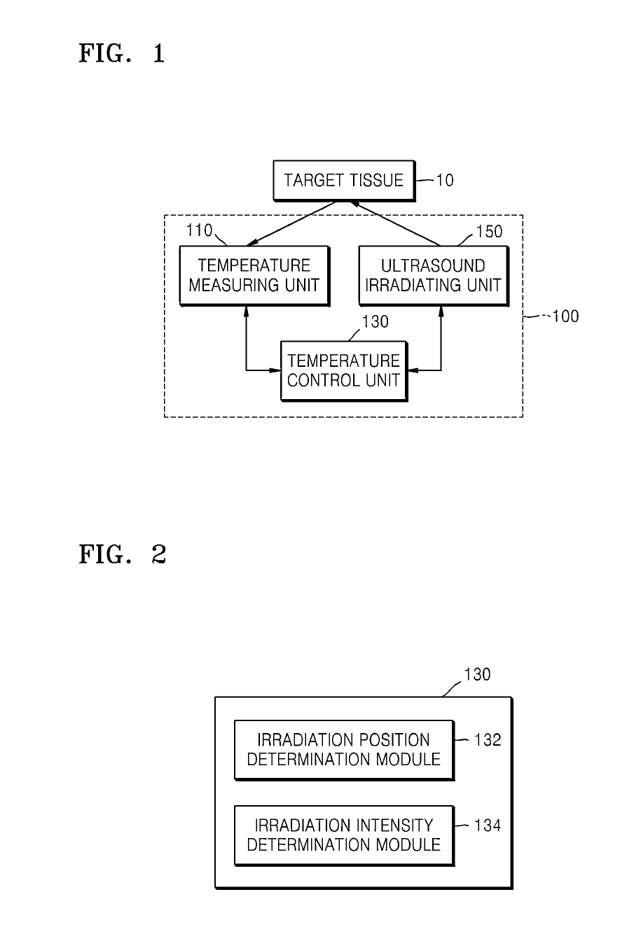

FIG. 1 is a block diagram illustrating a structure of a temperature controlling apparatus 100 according to an exemplary embodiment. It would be understood by one of ordinary skill in the art that other general-use components instead of or in addition to components illustrated in FIG. 1 may be further included.

Referring to FIG. 1, the temperature controlling apparatus 100 according to the current exemplary embodiment may include a temperature measuring unit 110 (e.g., temperature measurer), a temperature control unit 130 (e.g., temperature controller), and an ultrasound irradiating unit 150 (e.g., ultrasound irradiator).

The temperature measuring unit 110 measures a temperature of a target tissue 10. The temperature measuring unit 110 may measure a temperature of the entire target tissue 10 in real-time. The target tissue 10 may be living tissue including a lesion, such as a tumor. Examples of the temperature measuring unit 110 include not only a temperature measuring unit that is inserted into a body to measure a temperature of the target tissue 10, such as a thermocouple, but also a unit that is capable of measuring a temperature of the target tissue 10 by using a non-contact type imaging method such as ultrasound thermometry.

The temperature control unit 130 determines an intensity of ultrasound to be irradiated to the target tissue 10 based on a target temperature with respect to the target tissue 10 and a temperature of the target tissue 10 measured by using the temperature measuring unit 110. That is, the temperature control unit 130 may determine an optimum intensity of ultrasound irradiation for each predetermined unit time so that the target tissue 10 is maintained at a predetermined temperature for treatment. The temperature controlling apparatus 100 includes the temperature control unit 130 in order to, by using thermal energy due to ultrasound irradiation, change the target tissue 10 to be at a target temperature, at which treatment is possible. The target temperature may vary depending on a treatment method with respect to the target tissue 10. For example, when a lesion of the target tissue 10 is treated by using hyperthermia, a temperature, at which safety of living tissue may be secured so as to prevent destruction of the target tissue 10, may be set as a target temperature.

The ultrasound irradiating unit 150 irradiates ultrasound to the target tissue 10. The ultrasound irradiating unit 150 may include, for example, a transducer that generates and irradiates ultrasound and a driver that drives the transducer. When an intensity of ultrasound irradiation that is determined by using the temperature control unit 130 is input to the driver of the ultrasound irradiating unit 150, ultrasound to be irradiated to the target tissue 10 may be generated by using the transducer. Also, when information about a position to which ultrasound is to be irradiated is input to the driver of the ultrasound irradiating unit 150 by using the temperature control unit 130, ultrasound may also be irradiated to a desired position on the target tissue 10.

The temperature control unit 130 and the ultrasound irradiating unit 150 may repeatedly operate so that a temperature measured by using the temperature measuring unit 110 reaches the target temperature with respect to the target tissue 10. That is, the temperature control unit 130 and the ultrasound irradiating unit 150 may determine and irradiate an optimum ultrasound intensity for each predetermined unit time until the target tissue 10 reaches a predetermined temperature for treatment, and after the target tissue 10 has reached the target temperature, the temperature control unit 130 and the ultrasound irradiating unit 150 may determine an optimum ultrasound intensity for each predetermined unit time and irradiate corresponding ultrasound in order to maintain the target temperature. The predetermined unit time may be several to several hundreds of milliseconds or less.

Hereinafter, the temperature control unit 130 will be described in detail with reference to FIG. 2.

FIG. 2 is a diagram to describe the temperature control unit 130 of the temperature controlling apparatus 100, according to an exemplary embodiment. It would have been understood by one of ordinary skill in the art that other general-use components instead of or in addition to components illustrated in FIG. 2 may be further included.

The temperature control unit 130 may determine an intensity of ultrasound irradiation by applying a difference between a target temperature with respect to the target tissue 10 and a temperature of the target tissue 10 measured by using the temperature measuring unit 110 to a bio heat transfer model. As illustrated in FIG. 2, the temperature control unit 130 may include an irradiation position determination module 132 and an irradiation intensity determination module 134.

The irradiation position determination module 132 determines a position of a portion of the target tissue 10, to which ultrasound is to be irradiated. The irradiation position determination module 132 may determine a position of a portion of the target tissue 10 to which ultrasound is to be irradiated, for each predetermined unit time so that the entire area of the target tissue 10 is maintained at a target temperature. In the case of high intensity focused ultrasound (HIFU), ultrasound may not be simultaneously irradiated over the entire area of the target tissue 10 to increase a temperature of the target tissue 10. Thus, a wide area ultrasound irradiation whereby ultrasound is irradiated by changing portions of the target tissue 10 for irradiation for each predetermined unit time may be performed. A similar effect as if ultrasound is irradiated over the entire area of the target tissue 10 may be obtained when each predetermined unit time for irradiating ultrasound to a determined position of a portion of the target tissue 10 is very short. In other words, as the predetermined unit time during which an ultrasound irradiation position with respect to the target tissue 10 becomes shorter, the speed at which the ultrasound irradiation position is changed may increase, and accordingly, the temperature of the entire target tissue 10 may be maintained.

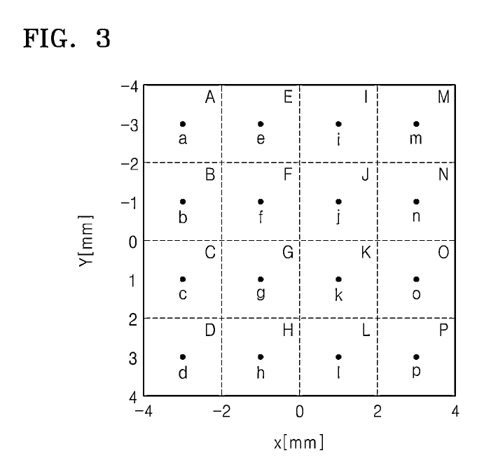

The irradiation position determination module 132 may determine at least one position of a portion of the target tissue 10, to which ultrasound is to be irradiated, for each predetermined unit time. That is, the irradiation position determination module 132 may determine one point or at least two points within the target tissue 10 for each predetermined unit time, as a position to which ultrasound is to be irradiated. For example, the ultrasound irradiating unit 150 may include a plurality of foci on which ultrasound is focused with respect to the target tissue 10, and the irradiation position determination module 132 may determine at least one of the positions of the plurality of foci as a position to which ultrasound is to be irradiated. Hereinafter, a method of determining a position of a portion of the target tissue 10, to which ultrasound is to be irradiated, by using the irradiation position determination module 132, will be described with reference to FIG. 3.

FIG. 3 illustrates a method of determining a position of a portion of a target tissue, to which ultrasound is to be irradiated, the method being performed by the irradiation position determination module 132 of the temperature control unit 130, according to an exemplary embodiment.

Referring to FIG. 3, a target tissue 10 having an area of 8 mm×8 mm is illustrated. As illustrated in FIG. 3, the target tissue 10 may be divided into sixteen equal areas, and ultrasound may be focused on a center of each area. That is, the target tissue 10 may be divided into a total of sixteen areas with an area of 2 mm×2 mm, that is, areas A to P, and a total of sixteen foci, foci a through p, may be respectively formed at centers of the respective areas. Each area is affected by ultrasound that is focused on a focus located at a center of each area. That is, when ultrasound is focused on focus a, area A mainly receives thermal energy due to the ultrasound, and a temperature of area A increases accordingly.

The irradiation position determination module 132 of the temperature control unit 130 may determine at least one of the positions of a plurality of foci where ultrasound is focused with respect to the target tissue 10 as a position where ultrasound is to be irradiated.

The irradiation position determination module 132 may determine an ultrasound irradiation position in consideration of temperatures at the respective positions of the plurality of foci. For example, the irradiation position determination module 132 may determine a position of a focus having a lowest temperature from among temperatures of positions of the plurality of foci, as an ultrasound irradiation position. That is, when a target temperature is 42° C., and temperatures of foci a through o are each 40° C., and a temperature of focus p is 38° C., focus p may be determined as an ultrasound irradiation position.

The irradiation position determination module 132 may also determine an ultrasound irradiation position by considering an average temperature of the target tissue 10 that is obtained by considering weights for distances from the positions of the plurality of respective foci on the target tissue 10 to an arbitrary position on the target tissue 10. For example, the irradiation position determination module 132 may calculate an average temperature of the target tissue 10 by applying a lower weight as a distance from the respective positions of the foci on the target tissue 10 to an arbitrary position on the target tissue 10 becomes greater and may determine an ultrasound irradiation position by considering the average temperature of the target tissue 10. The irradiation position determination module 132 may also determine an ultrasound irradiation position by applying a weight of 0 to other positions than an area, in which each focus is included, and calculating an average temperature of each area by applying a weight according to distance, only to a position within an area of the target tissue 10 in which each focus is included. That is, for focus a, an ultrasound irradiation position may be determined with respect to a weighted average temperature that is calculated by applying a weight according to a distance between focus a and an arbitrary position within area A; also, for foci b through p, an ultrasound irradiation position may be determined with respect to each weighted average temperature that is calculated in the above manner.

The irradiation position determination module 132 may determine at least two foci at the same value as ultrasound irradiation positions. That is, according to the above-described method, when at least two foci have the same value, all of the at least two foci may be determined as ultrasound irradiation positions. For example, from among foci a through p, if temperatures or average temperatures of focus f and focus k are the lowest, both focus f and focus k may be determined as ultrasound irradiation positions. Moreover, when a first priority focus and a second priority focus are determined according to the above-described method, both the first priority focus and the second priority focus may be determined as ultrasound irradiation positions. For example, when a temperature or an average temperature of focus g from among foci a through p is the lowest, and a temperature or an average temperature of focus j is next to the lowest, both focus g and focus j may be determined as ultrasound irradiation positions.

Referring to FIG. 2 again, the irradiation intensity determination module 134 determines an intensity of ultrasound irradiation with respect to the determined positions based on a target temperature with respect to the target tissue 10 and a temperature of a portion of the target tissue 10 measured with respect to the position determined by using the irradiation position determination module 132. Here, an intensity of ultrasound irradiation with respect to the position determined by using the irradiation position determination module 132 may be determined by using a bio heat transfer model.

A bio heat transfer model is a mathematical expression of a change in a temperature of a predetermined tissue and may be expressed as in Equation 1 below.

$${ρ}_{t} \cdot {C}_{t} \cdot \frac{\partial T ( x , t )}{\partial t} = {k}_{t} \cdot {∇}^{2} T ( x , t ) + {V}_{{ρ}_{b}} \cdot {C}_{b} \cdot ( {T}_{b} - T ( x , t ) ) + Q ( x , t ) , \qquad [ Equation 1 ]$$

where ρₜ denotes a density of a tissue, Cₜ is a specific heat of the tissue. T(x,t) is a temperature at a position X in a tissue and at a time t,

$$\frac{\partial T ( x , t )}{\partial t}$$

denotes a variation of T(x,t) with respect to a time t and indicates a primary differential value with respect to a time t, and Δ²T(x,t) denotes a variation with respect to a position T(x,t) and indicates a secondary spatial differential value with respect to a position X. kₜ denotes a thermal conductivity of a tissue, V_{ρ}_{ₕ} denotes a perfusion rate of hematocele in a tissue, C_{b} is a specific heat of hematocele in a tissue, and T_{b} denotes a temperature of hematocele in a tissue. Q(x,t) denotes heat applied from the outside to a position X in a tissue at a time t.

Heat (Q(x,t) ) that is applied from the outside to a position X in a tissue at a time t may be applied by using various heat generators. For example, heat may be applied to a tissue by irradiating ultrasound; here, heat (Q) that is applied to a tissue by irradiating ultrasound may be expressed as in Equation 2 below.

Q=2·β·f·I, [Equation 2]

where β denotes an absorption coefficient of a tissue, f denotes a frequency of ultrasound, and I denotes an intensity of ultrasound irradiation.

When heat is applied to a tissue by irradiating ultrasound, an intensity of ultrasound irradiation may be expressed as in Equation 3 below by using Equations 1 and 2.

$$I = \frac{\frac{{ρ}_{t} \cdot {C}_{t} \cdot \frac{\partial T ( x , t )}{\partial t} - {k}_{t} \cdot {∇}^{2} T ( x , t ) -}{{V}_{{ρ}_{b}} \cdot {C}_{b} \cdot ( {T}_{b} - T ( x , t ) )}}{2 \cdot β \cdot f \cdot 10} \qquad [ Equation 3 ]$$

The temperature controlling apparatus 100 according to the current exemplary embodiment may determine an intensity of ultrasound irradiation at which a target temperature may be reached, by applying, as a difference between a target temperature and a measured temperature, a value of

$$\frac{\partial T ( x , t )}{\partial t}$$

denoting a primary differential value with respect to a time t as a variation with respect to a time T(x,t) of Equation 3.

When there are at least two positions to which ultrasound is to be irradiated, the irradiation intensity determination module 134 may determine an intensity of ultrasound irradiation with respect to the determined positions, based on a temperature that is closest to a target temperature, from among temperatures measured with respect to the positions. For example, when a target temperature is 42° C., and temperatures of foci a through n are all 40° C., and a temperature of focus o is 37° C., and a temperature of focus p is 38° C., 38° C., which is the temperature of focus p, may be applied when determining an intensity of ultrasound irradiation with respect to focus n and focus o. When two positions to which ultrasound is to be irradiated are determined by using the irradiation position determination module 132, and temperatures or average temperatures at the two positions are the same, a single, measured temperature is to be applied to a bio heat transfer model, and thus, the single, measured temperature may be applied. However, when a first priority position and a second priority position are both determined as positions to which ultrasound is to be irradiated, an intensity of ultrasound irradiation may be determined by applying a temperature, or an average temperature of the second priority position may be applied as a measured temperature to be applied to a bio heat transfer model. This is in order to prevent an excess of a target temperature at any one of the two positions even if ultrasound of the determined irradiation intensity is irradiated to the two positions. If the target temperature is exceeded, the target tissue 10 might be destroyed.

Alternatively, when there are at least two positions to which ultrasound is to be irradiated, the irradiation intensity determination module 134 may determine an intensity of ultrasound irradiation with respect to each of the two positions based on temperatures respectively measured regarding the determined positions. For example, in the above example, for focus o, an intensity of ultrasound irradiation may be determined based on 37° C., which is a measured temperature thereof, and for focus p, an intensity of ultrasound irradiation may be determined based on 38° C., which is a measured temperature thereof.

Alternatively with respect to the irradiation intensity determination module 134, when there are at least two positions to which ultrasound is to be irradiated, the irradiation intensity determination module 134 may determine an intensity of ultrasound irradiation within a safe range, with respect to each of the determined positions, based on a temperature that is most different from the target temperature from among temperatures respectively measured with respect to the determined positions. In the above example, an intensity of ultrasound irradiation may be determined based on 37° C., which is a temperature of focus n; however, the intensity of ultrasound irradiation that is determined here has to be within a safe range with respect to each of the determined positions.

In addition, the irradiation position determination module 132 may also determine an ultrasound irradiation position by using a bio heat transfer model. The irradiation position determination module 132 may determine an ultrasound irradiation position by predicting a temperature distribution of the target tissue 10 by applying a temperature measured with respect to the target tissue 10 and ultrasound of a predetermined irradiation intensity to a bio heat transfer model. That is, when ultrasound is focused on a predetermined position of the target tissue 10, heat is transferred in the target tissue 10 through heat diffusion, and the irradiation position determination module 132 may predict a temperature distribution of the target tissue 10 and determine an optimum position by using a bio heat transfer model in consideration of the heat diffusion.

An ultrasound irradiation position determined by using the irradiation position determination module 132 and an intensity of ultrasound irradiation determined by using the irradiation intensity determination module 134 are transferred to the ultrasound irradiating unit 150, and the ultrasound irradiating unit 150 may generate ultrasound of the determined intensity of ultrasound irradiation and irradiate the same to the determined ultrasound irradiation position by reflecting the ultrasound irradiation position and the intensity of ultrasound irradiation.

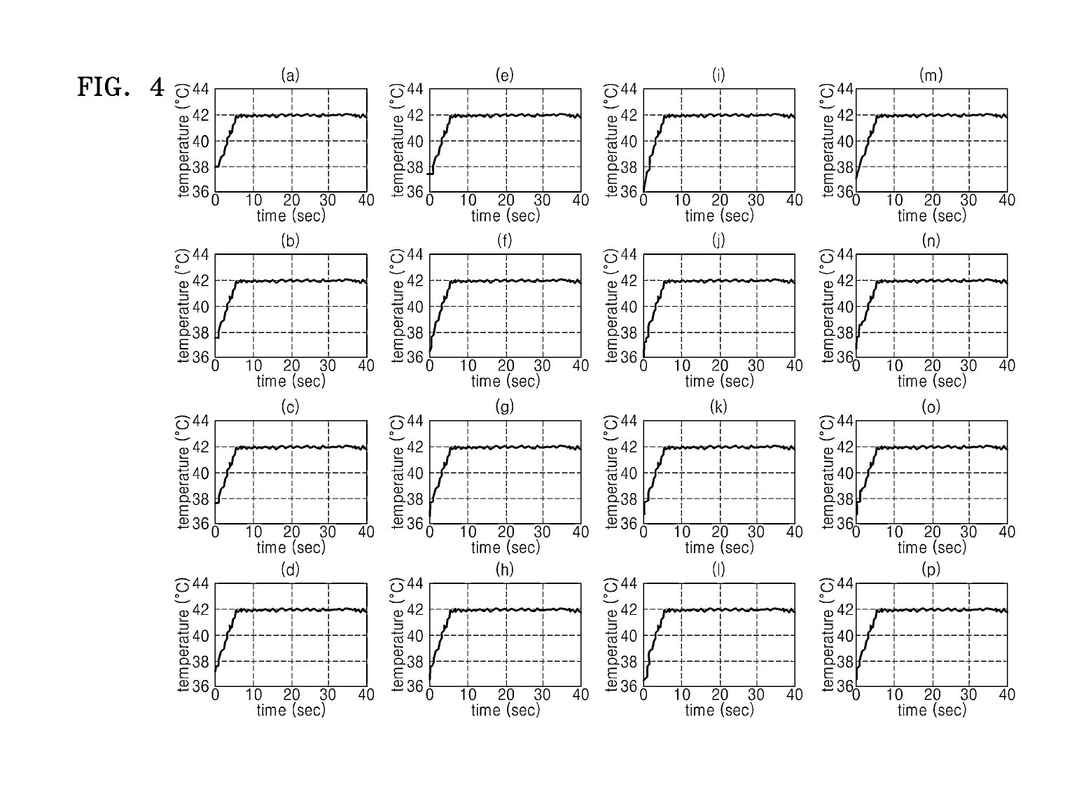

FIG. 4 shows graphs showing a change in temperature of a plurality of foci on which ultrasound is focused with respect to a target tissue, according to time, when a temperature control with respect to the target tissue is performed by using a temperature controlling apparatus according to an exemplary embodiment.

Referring to FIG. 4, temperature changes in a total of sixteen foci according to time, from focus a through focus p located within the target tissue 10 described with reference to FIG. 3, are shown. That is, FIG. 4 shows temperature changes occurring at the respective foci for 40 seconds when irradiation is irradiated to the target tissue 10 for each predetermined unit time by setting a target temperature of the temperature controlling apparatus 100 at 42° C.

As the temperature controlling apparatus 100 irradiates ultrasound by determining an ultrasound irradiation position and an intensity of ultrasound irradiation with respect to the target tissue 10 for each predetermined unit time, ultrasound may be irradiated to at least one of a total of sixteen foci, from focus a to focus p, for each predetermined unit time. Referring to FIG. 4, temperatures at all foci approach close to 42° C., which is a target temperature within 10 seconds, and are maintained at the target temperature until 40 seconds. The temperature controlling apparatus 100 may keep a temperature of the entire area of the target tissue 10 at the target temperature by irradiating ultrasound of optimum ultrasound intensity to at least one position of a portion of the target tissue 10 for a predetermined unit time during 40 seconds.

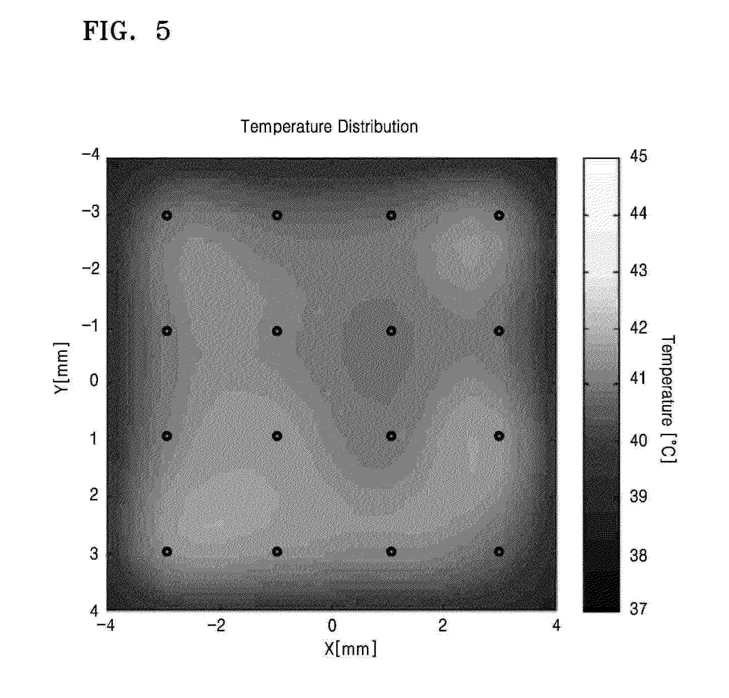

FIG. 5 illustrates a temperature distribution with respect to a target tissue when a temperature control with respect to the target tissue is performed by using a temperature controlling apparatus according to an exemplary embodiment.

Referring to FIG. 5, a temperature distribution with respect to the target tissue 10 after a predetermined period of time has passed is shown when a temperature of the target tissue 10 is controlled by irradiating ultrasound to the target tissue 10 by using the temperature controlling apparatus 100. That is, FIG. 5 shows a temperature distribution after a predetermined period of time when ultrasound is irradiated to the target tissue 10 for each predetermined unit time by setting a target temperature of the temperature controlling apparatus 100 at 42° C. Compared to FIGS. 3 and 4, the temperature distribution of FIG. 5 may be a temperature distribution of the target tissue 10 that is obtained by setting a target temperature of 42° C. and 40 seconds later after irradiating ultrasound of an optimum irradiation intensity to at least one of the total of sixteen foci of the target tissue 10, from focus a to focus p. Referring to FIG. 5, the entire area of the target tissue 10 is maintained at a temperature close to 42° C., which is the target temperature.

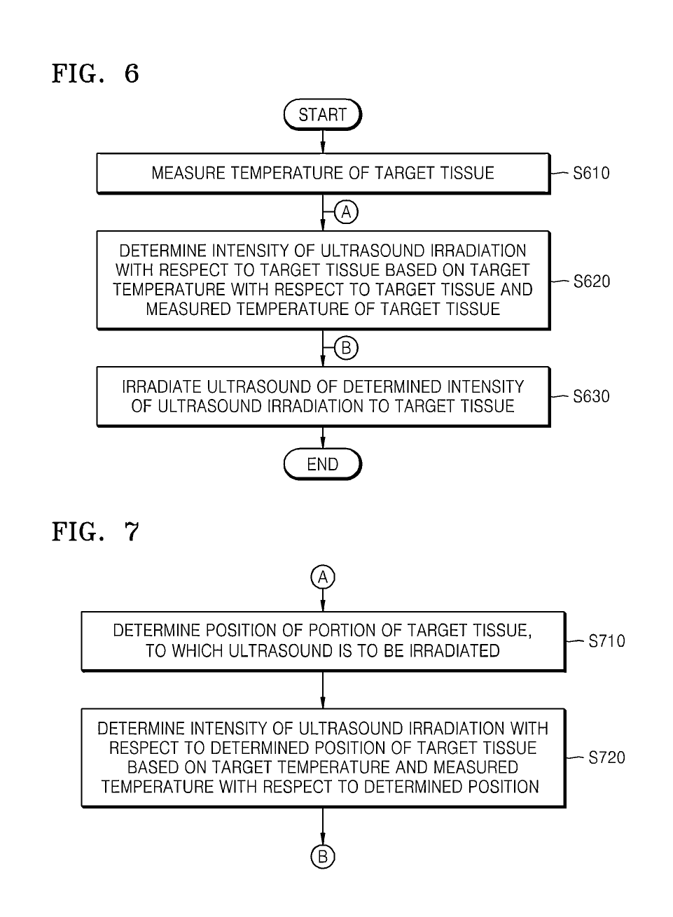

FIG. 6 is a flowchart of a method of controlling a temperature of tissue, according to an exemplary embodiment. The temperature controlling apparatus 100 according to an exemplary embodiment may also be applied to the method of FIG. 6.

In operation 610, the temperature measuring unit 110 measures a temperature of the target tissue 10. The temperature measuring unit 110 may measure a temperature of the entire target tissue 10 in real-time.

In operation 620, the temperature control unit 130 determines an intensity of ultrasound irradiation with respect to the target tissue 10 based on a target temperature and a measured temperature with respect to the target tissue 10. An intensity of ultrasound irradiation may be determined by applying a difference between a target temperature and a measured temperature with respect to the target tissue 10 to a bio heat transfer model. Hereinafter, an operation of determining an intensity of ultrasound irradiation will be described in detail with reference to FIG. 7.

FIG. 7 is a detailed flowchart of an operation of determining an intensity of ultrasound irradiation in a method of controlling a temperature of tissue, according to an exemplary embodiment.

In operation 710, the irradiation position determination module 132 of the temperature control unit 130 determines a position of a portion of the target tissue 10, to which ultrasound is to be irradiated. The irradiation position determination module 132 may determine at least one position of a portion of the target tissue 10, to which ultrasound is to be irradiated, for each predetermined unit time. For example, the irradiation position determination module 132 may determine at least one of positions of a plurality of foci on which ultrasound is focused with respect to the target tissue 10, as an ultrasound irradiation position.

The irradiation position determination module 132 may determine an ultrasound irradiation position in consideration of temperatures at the respective positions of the plurality of foci. For example, the irradiation position determination module 132 may determine a position of a focus having a lowest temperature from among temperatures of positions of the plurality of foci, as an ultrasound irradiation position.

The irradiation position determination module 132 may also determine an ultrasound irradiation position by considering an average temperature of the target tissue 10 that is obtained by considering weights for distances from the positions of the plurality of respective foci on the target tissue 10 to an arbitrary position on the target tissue 10. For example, the irradiation position determination module 132 may calculate an average temperature of the target tissue 10 by applying a lower weight as a distance from the respective positions of the foci on the target tissue 10 to an arbitrary position on the target tissue 10 becomes greater and may determine an ultrasound irradiation position by considering the average temperature of the target tissue 10. Here, the irradiation position determination module 132 may also determine an ultrasound irradiation position by applying a weight of 0 to other positions than an area, in which each focus is included, and calculating an average temperature of each area by applying a weight according to distance, only to a position within an area of the target tissue 10 in which each focus is included.

The irradiation position determination module 132 may determine at least two foci at the same value as ultrasound irradiation positions. That is, according to the above-described method, when the at least two foci have the same value, both of the foci may be determined as positions to which ultrasound is to be irradiated. Also, when a first priority focus and a second priority focus are determined according to the above-described method, both the first priority focus and the second priority focus may be determined as ultrasound irradiation positions.

The irradiation position determination module 132 may also determine an ultrasound irradiation position by using a bio heat transfer model. The irradiation position determination module 132 may determine an ultrasound irradiation position by predicting a temperature distribution of the target tissue 10 by applying ultrasound of a predetermined irradiation intensity to a bio heat transfer model.

In operation 720, the irradiation intensity determination module 134 of the temperature control unit 130 determines an intensity of ultrasound irradiation with respect to a determined position based on a target temperature and a temperature measured with respect to the position determined by using the irradiation position determination module 132. According to the method of controlling a temperature of the exemplary embodiments, an intensity of ultrasound irradiation with respect to a position determined by using the irradiation position determination module 132 may be determined by applying a difference between a target temperature and a measured temperature to a bio heat transfer model.

When there are at least two ultrasound irradiation positions, the irradiation intensity determination module 134 may determine an intensity of ultrasound irradiation with respect to the determined positions based on a temperature that is closest to the target temperature from among temperatures respectively measured with respect to the determined positions.

Alternatively with respect to the irradiation intensity determination module 134, when there are at least two ultrasound irradiation positions, the irradiation intensity determination module 134 may determine an intensity of ultrasound irradiation with respect to each of the determined positions based on temperatures respectively measured with respect to the determined positions.

Also, alternatively with respect to the irradiation intensity determination module 134, when there are at least two ultrasound irradiation positions, the irradiation intensity determination module 134 may determine an intensity of ultrasound irradiation with respect to each of the determined positions within a safe range, with respect to each of the determined positions, based on a temperature that is most different from the target temperature from among temperatures respectively measured with respect to the determined positions.

Referring to FIG. 6 again, in operation 630, the ultrasound irradiating unit 150 irradiates to the target tissue 10 ultrasound of an irradiation intensity that is determined by using the irradiation intensity determination module 134 of the temperature control unit 130. An ultrasound irradiation position determined by using the irradiation position determination module 132 of the temperature control unit 130 and an intensity of ultrasound irradiation determined by using the irradiation intensity determination module 134 of the temperature control unit 130 are transmitted to the ultrasound irradiating unit 150. By reflecting the ultrasound irradiation position and the intensity of ultrasound irradiation, the ultrasound irradiating unit 150 may generate ultrasound having the determined intensity of ultrasound irradiation and irradiate the ultrasound to the determined ultrasound irradiation position.

Operation 620 of determining an intensity of ultrasound irradiation and operation 630 of irradiating ultrasound to the target tissue 10 may be repeated for each predetermined unit time until the measured temperature reaches a target temperature with respect to the target tissue 10.

As described above, according to the one or more of the above exemplary embodiments, when treating a target tissue by using focused ultrasound, an optimum ultrasound intensity for maintaining the target tissue at a target temperature is determined and ultrasound of the optimum ultrasound intensity is irradiated to the target tissue, thereby accurately controlling a temperature of the target tissue within a short time.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope of the exemplary embodiments as defined by the following claims.

What is claimed is:

1. A method of controlling a temperature of tissue, the method comprising:
measuring a temperature of a target tissue;
determining an intensity of ultrasound irradiation with respect to the target tissue based on a target temperature of the target tissue and the measured temperature of the target tissue; and
irradiating the ultrasound irradiation having the determined intensity to the target tissue.

2. The method of claim 1, wherein the determining of the intensity of the ultrasound irradiation comprises applying a difference between the target temperature of the target tissue and the measured temperature to a bio heat transfer model to thereby determine the intensity of the ultrasound irradiation.

3. The method of claim 1, further comprising determining an ultrasound irradiation position of a portion of the target tissue, to which the ultrasound irradiation is to be irradiated,
wherein the determining of the intensity of the ultrasound irradiation comprises determining an intensity of ultrasound irradiation with respect to the determined ultrasound irradiation position based on the target temperature and the measured temperature with respect to the determined position.

4. The method of claim 3, wherein the determining of the ultrasound irradiation position comprises determining at least one position of positions of a plurality of foci on which ultrasound is focused with respect to the target tissue as the ultrasound irradiation position.

5. The method of claim 4, wherein the determining of the ultrasound irradiation position comprises determining the ultrasound irradiation position according to temperatures of the positions of the plurality of foci.

6. The method of claim 5, wherein the determining of the ultrasound irradiation position comprises determining a position of a focus at a lowest temperature from among temperatures of the positions of the plurality of foci as the ultrasound irradiation position.

7. The method of claim 4, wherein the determining of the ultrasound irradiation position comprises determining the ultrasound irradiation position according to an average temperature of the target tissue that is obtained according to weights corresponding to distances from the positions of the plurality of respective foci on the target tissue to an arbitrary position on the target tissue.

8. The method of claim 7, wherein the determining of the ultrasound irradiation position comprises calculating the average temperature of the target tissue by applying a smaller weight as a distance from the respective positions of the foci on the target tissue to the arbitrary position on the target tissue becomes greater and applying a greater weight which is greater than the smaller weight as the distance from the respective positions of the foci on the target tissue to the arbitrary position on the target tissue becomes smaller.

9. The method of claim 3, wherein the determining of the intensity of ultrasound irradiation comprises, when there are at least two ultrasound irradiation positions, determining an intensity of ultrasound irradiation with respect to the at least two ultrasound irradiation positions based on a temperature that is closest to the target temperature from among temperatures respectively measured with respect to the at least two ultrasound irradiation positions.

10. The method of claim 3, wherein the determining of the intensity of ultrasound irradiation comprises, when there are at least two ultrasound irradiation positions, determining an intensity of ultrasound irradiation with respect to each of the at least two ultrasound irradiation positions based on temperatures respectively measured with respect to the at least two ultrasound irradiation positions.

11. The method of claim 3, wherein the determining of the intensity of ultrasound irradiation comprises, when there are at least two ultrasound irradiation positions, determining an intensity of ultrasound irradiation with respect to each of the at least two ultrasound irradiation positions within a safe range, with respect to each of the at least two ultrasound irradiation positions, based on a temperature that is most different from the target temperature from among temperatures respectively measured with respect to the at least two ultrasound irradiation positions.

12. The method of claim 1, wherein the determining of the intensity of ultrasound irradiation and the irradiating of the ultrasound irradiation to the target tissue are repeatedly performed for each of a plurality of predetermined units of time so that the measured temperature reaches the target temperature with respect to the target tissue.

13. A temperature controlling apparatus comprising:
a temperature measurer configured to measure a temperature of the target tissue;
a temperature controller configured to determine an intensity of ultrasound irradiation with respect to the target tissue based on a target temperature of the target tissue and the measured temperature of the target tissue; and
an ultrasound irradiator configured to irradiate the ultrasound irradiation having the determined intensity to the target tissue.

14. The temperature controlling apparatus of claim 13, wherein the temperature controller is configured to determine the intensity of the ultrasound irradiation by applying a difference between the target temperature and the measured temperature to a bio heat transfer model to thereby determine the intensity of the ultrasound irradiation.

15. The temperature controlling apparatus of claim 13, wherein the temperature controller comprises:
an irradiation position determination module configured to determine an ultrasound irradiation position of a portion of the target tissue, to which the ultrasound irradiation is to be irradiated; and
an irradiation intensity determination module configured to determine an intensity of ultrasound irradiation with respect to the determined position, based on the target temperature and a temperature measured with respect to the determined ultrasound irradiation position.

16. The temperature controlling apparatus of claim 15, wherein the irradiation position determination module is configured to determine at least one position of positions of a plurality of foci on which the ultrasound irradiation is focused with respect to the target tissue, as the ultrasound irradiation position.

17. The temperature controlling apparatus of claim 16, wherein the irradiation position determination module is configured to determine the ultrasound irradiation position according to temperatures of the positions of the plurality of foci.

18. The temperature controlling apparatus of claim 17, wherein the irradiation position determination module is configured to determine a position of a focus at a lowest temperature from among temperatures of the positions of the plurality of foci, as the ultrasound irradiation position.

19. The temperature controlling apparatus of claim 16, wherein the irradiation position determination module is configured to determine the ultrasound irradiation position by considering an average temperature of the target tissue that is obtained by considering weights corresponding to distances from the positions of the plurality of respective foci on the target tissue to an arbitrary position on the target tissue.

20. The temperature controlling apparatus of claim 19, wherein the irradiation position determination module is configured to calculate the average temperature of the target tissue by applying a lower weight as a distance from the respective positions of the foci on the target tissue to the arbitrary position on the target tissue becomes greater and applying a greater weight which is greater than the smaller weight as the distance from the respective positions of the foci on the target tissue to the arbitrary position on the target tissue becomes smaller.

21. The temperature controlling apparatus of claim 15, wherein when there are at least two ultrasound irradiation positions, the irradiation intensity determination module is configured to determine an intensity of ultrasound irradiation with respect to the at least two ultrasound irradiation positions based on a temperature that is closest to the target temperature from among temperatures measured with respect to the at least two ultrasound irradiation positions.

22. The temperature controlling apparatus of claim 15, wherein when there are at least two ultrasound irradiation positions, the irradiation intensity determination module is configured to determine an intensity of ultrasound irradiation with respect to each of the at least two ultrasound irradiation positions based on temperatures respectively measured with respect to the at least two ultrasound irradiation positions.

23. The temperature controlling apparatus of claim 15, wherein when there are at least two ultrasound irradiation positions, the irradiation intensity determination module is configured to determine an intensity of ultrasound irradiation with respect to each of the at least two ultrasound irradiation positions within a safe range, with respect to each of the at least two ultrasound irradiation positions, based on a temperature that is most different from the target temperature from among temperatures respectively measured with respect to the at least two ultrasound irradiation positions.

24. The temperature controlling apparatus of claim 13, wherein the temperature control unit and the ultrasound irradiating unit are configured to repeatedly operate so that the measured temperature reaches the target temperature with respect to the target tissue.
